(19) United States
(12) Patent Application Publication
(10) Pub. No.: US 2008/0243242 A1
(43) Pub. Date: Oct. 2, 2008

(54) METHOD FOR PRODUCING A CORROSION-INHIBITING COATING ON AN IMPLANT MADE OF A BIO-CORRODIBLE MAGNESIUM ALLOY AND IMPLANT PRODUCED ACCORDING TO THE METHOD

(75) Inventors: Gerhard Kappelt, Uttenreuth (DE); Peter Kurze, Nideggen (DE); Dora Banerjee, Kerpen (DE)

Correspondence Address: POWELL GOLDSTEIN LLP, ONE ATLANTIC CENTER FOURTEENTH FLOOR, 1201 WEST PEACHTREE STREET NW, ATLANTA, GA, 30309-3488, US

(73) Assignee: BIOTRONIK VI PATENT AG, Baar (CH)

(21) Appl. No.: 11/957,512

(22) Filed: Dec. 17, 2007

(30) Foreign Application Priority Data
Dec. 19, 2006 (DE) 10 2006 060 501.2

(51) Int. Cl. A61L 27/30; A61L 27/04; C25D 11/34; C25D 11/04; A61F 2/82

(52) U.S. Cl. 623/1.46; 427/2.25; 205/151

(57) ABSTRACT

A method for producing a corrosion-inhibiting coating on an implant made of a biocorrodible magnesium alloy, the method comprising providing the implant; and treating the implant surface using an aqueous or alcoholic conversion solution containing one or more ions selected from the group consisting of K⁺, Na⁺, NH₄⁺, Ca²⁺, Mg²⁺, Zn²⁺, Ti⁴⁺, Zr⁴⁺, Ce³⁺, Ce⁴⁺, PO₄³⁻, HPO₄²⁻, H₂PO₄⁻, OH⁻, B₃³⁻, B₄O₇²⁻, SiO₃²⁻, MnO₄²⁻, MnO₄⁻, VO₃⁻, WO₄²⁻, MoO₄²⁻, TiO₃²⁻, Se²⁻, ZrO₃²⁻, and NbO₄⁻, wherein the concentration of the ion or ions is in the range of from 0.01 mol/l to 2 mol/l. An implant produced by this method is also disclosed.

### FIELD

The present invention relates to a method for producing a corrosion-inhibiting coating on an implant made of a biocorrodible magnesium alloy and implants obtained or obtainable according to the method.

### BACKGROUND

Medical implants of greatly varying intended purposes are known in a great manifold in the prior art. It is frequently only necessary for the implant to remain in the body temporarily to fulfill the medical purpose. Implants made of permanent materials, i.e., materials which are not degraded in the body, are to be removed again because rejection reactions of the body may occur in the moderate and long term even with high biocompatibility.

One approach for avoiding a further surgical intervention comprises molding the implant entirely or partially from a biocorrodible material. For purposes of the present disclosure, biocorrosion refers to microbial procedures or processes solely caused by the presence of body media which result in a gradual degradation of the structure comprising the material. At a specific point in time, the implant or at least the part of the implant which comprises the biocorrodible material loses mechanical integrity. The degradation products are largely resorbed by the body. These products, such as magnesium, for example, may even unfold a positive therapeutic effect locally. Small quantities of non-resorbable degradation products are tolerable.

Biocorrodible materials have been developed, inter alia, on the basis of polymers of a synthetic nature or a natural origin. The mechanical material properties (low plasticity) and the low biocompatibility of the degradation products of the polymers (partially elevated thrombogenesis, increased inflammation) sometimes significantly limit the use, however. Thus, for example, orthopedic implants must frequently withstand high mechanical strains and vascular implants, such as stents, must meet very special requirements for modulus of elasticity, brittleness, and deformability.

A promising approach for solving the problem is the use of biocorrodible metal alloys. Thus, it was suggested in German Patent Application No. 197 31 021 A1 that medical implants be molded from a metallic material whose main component is an element from the group consisting of alkali metals, alkaline earth metals, iron, zinc, and aluminum. Alloys based on magnesium, iron, and zinc are described as especially suitable. Secondary components of the alloys may be manganese, cobalt, nickel, chromium, copper, cadmium, lead, tin, thorium, zirconium, silver, gold, palladium, platinum, silicon, calcium, lithium, aluminum, zinc, and iron. Furthermore, the use of a biocorrodible magnesium alloy having a proportion of magnesium >90%, yttrium 3.7-5.5%, rare earth metals 1.5-4.4%, and the remainder <1% is known from German Patent Application No. 102 53 634 A1, which is suitable, in particular, for producing an endoprosthesis, e.g., in the form of a self-expanding or balloon-expandable stent. Notwithstanding the progress achieved in the field of biocorrodible metal alloys, the currently known alloys are only usable in a restricted way because of their corrosion behavior. The relatively rapid biocorrosion of the magnesium alloys, in particular, in the field of structures which are strongly mechanically strained, limits the use of the magnesium alloys.

Both the fundamentals of magnesium corrosion and a large number of technical methods for improving the corrosion behavior (in the meaning of reinforcing the corrosion protection) are known in the prior art. It is known, for example, that the addition of yttrium and/or further rare earth metals to a magnesium alloy provides a slightly increased corrosion resistance in seawater.

One approach provides producing a corrosion-protecting layer on the molded body comprising magnesium or a magnesium alloy. Known methods for producing a corrosion-protecting layer have been developed and optimized from the aspect of a technical use of the molded body, but not a medical-technical use in biocorrodible implants in physiological surroundings. These known methods comprise the application of polymers or inorganic cover layers, the production of an enamel, the chemical conversion of the surface, hot gas oxidation, anodization, plasma spraying, laser beam remelting, PVD methods, ion implantation, or lacquering.

Typical technical areas of use of molded bodies made of magnesium alloys outside medical technology normally require extensive suppression of corrosive processes. Accordingly, the goal of most technical methods is complete inhibition of corrosive processes. In contrast, the goal of improving the corrosion behavior of biocorrodible magnesium alloys is not the complete suppression, but rather only the inhibition of corrosive processes. For this reason alone, most known methods are not suitable for producing a corrosion protection layer. Furthermore, toxicological aspects must also be taken into consideration for a medical technology use. Moreover, corrosive processes are also strongly a function of the medium in which they occur; and, therefore, it is not unrestrictedly possible to transfer the findings on corrosion protection obtained under typical environmental conditions in the technical field to the processes in a physiological environment. Finally, in manifold medical implants, the mechanisms on which the corrosion is based may also deviate from typical technical applications of the material. Thus, for example, stents, surgical suture material, or clips are mechanically deformed in use so that the partial process of tension cracking corrosion may have great significance in the degradation of these molded bodies.

German Patent Application No. 101 63 106 A1 provides changing the magnesium material in its corrosivity by modification with halogenides. The magnesium material is to be used for producing medical implants. The halogenide is preferably a fluoride. The material is modified by alloying halogen compounds in salt form. The composition of the magnesium alloy is accordingly changed by adding the halogenides to reduce the corrosion rate. Accordingly, the entire molded body comprising such a modified alloy will have an altered corrosion behavior. However, further material properties, which are significant in processing or also affect the mechanical properties of the molded body resulting from the material, may be influenced by the alloying.

### SUMMARY

The present disclosure describes several exemplary embodiments of the present invention.

One aspect of the present disclosure provides a method for producing a corrosion-inhibiting coating on an implant having a surface an made of a biocorrodible magnesium alloy, the method comprising a) treating the implant surface using an aqueous or alcoholic conversion solution comprising one or more ions selected from the group consisting of K⁺, Na⁺, NH₄⁺, Ca²⁺, Mg²⁺, Zn²⁺, Ti⁴⁺, Zr⁴⁺, Ce³⁺, Ce⁴⁺, PO₄³⁻, HPO₄²⁻, H₂PO₄⁻, OH⁻, BO₃³⁻, B₄O₇²⁻, SiO₃²⁻, MnO₄⁻, MnO₄⁻, VO₃⁻, WO₄²⁻, MoO₄²⁻, TiO₃²⁻, Se²⁻, ZrO₃²⁻, and NbO₄⁻, wherein the concentration of the ion or the ions is in the range of from 0.01 mol/l to 2 mol/l, respectively.

Another aspect of the present disclosure provides an implant having a corrosion-inhibiting coating provided by a method, comprising a) treating the implant surface using an aqueous or alcoholic conversion solution containing one or more ions selected from the group consisting of K⁺, Na⁺, NH₄⁺, Ca²⁺, Mg²⁺, Zn²⁺, Ti⁴⁺, Zr⁴⁺, Ce³⁺, Ce⁴⁺, PO₄³⁻, HPO₄²⁻, H₂PO₄⁻, OH⁻, BO₃³⁻, B₄O₇²⁻, SiO₃²⁻, MnO₄²⁻, MnO₄⁻, VO₃⁻, WO₄²⁻, MoO₄²⁻, TiO₃²⁻, Se²⁻, ZrO₃²⁻, and NbO₄⁻, wherein the concentration of the ion or the ions is in the range of from 0.01 mol/l to 2 mol/l, respectively.

The present disclosure provides an alternative or preferably improved method for producing a corrosion-inhibiting coating on an implant made of a biocorrodible magnesium alloy. The corrosion-inhibiting coating provided by the present disclosure causes a temporary inhibition, but not complete suppression, of the corrosion of the material in a physiological environment.

### DETAILED DESCRIPTION

It has been shown that the production of a coating in the disclosed method does not result in the implementation of a protective layer which completely or largely inhibits the corrosion in a physiological environment. In other words, corrosion of the implant still occurs in a physiological environment, but at significantly reduced speed. The treatment of the implant surface with the conversion solution causes an anodic oxidation of the implant. The treatment of the implant is either performed without use of an external power source (externally unpowered) or with a power source.

The corrosion-inhibiting coating accordingly arises through surface-proximal conversion of the material of the implant. There is thus no application of material to a surface of the implant, but rather a chemical conversion of the metallic surface and the various components of the conversion solution.

Of the listed ions, OH⁻ ions in an aqueous or alcoholic system fulfill a special function. They form a stable barrier layer made of Mg(OH)₂ on the surface of the implant, below a part of the conversion layer formed by the further ions. The barrier layer obstructs the diffusion of corrosion-encouraging ions into the metal and is highly ductile in the event of mechanical deformations. The conversion solution, therefore, preferably contains OH-ions and one or more ions selected from the group consisting of K⁺, Na⁺, NH₄⁺, Ca²⁺, Mg²⁺, Zn²⁺, Ti⁴⁺, Zr⁴⁺, Ce³⁺, Ce⁴⁺, PO₄³⁻, HPO₄²⁻, H₂PO₄⁻, OH⁻, BO₃³⁻, B₄O₇²⁻, SiO₃²⁻, MnO₄²⁻, MnO₄⁻, VO₃⁻, WO₄²⁻, MoO₄²⁻, TiO₃²⁻, Se²⁻, ZrO₃²⁻, and NbO₄⁻.

Cover layers having lower solubility form on the above-mentioned barrier layer, particularly from aqueous or alcoholic conversion solutions having the anions PO₄³⁻, H₂PO₄⁻, HPO₄²⁻, BO₃³⁻, B₄O₇²⁻ and SiO₃²⁻0 and thus additionally protect the implant. Moreover, these cover layers are also ductile so that they do not crack off upon mechanical deformation of the implant. The conversion solution, therefore, preferably contains OH⁻ ions and one or more anions selected from the group consisting of PO₄³⁻, H₂PO₄⁻, HPO₄²⁻, BO₃³⁻, B₄O₇²⁻, and SiO₃²⁻.

Of the cited cations, K⁺, Na⁺, NH₄⁺, Ca²⁺, and Mg²⁺ are already present in the body so that soluble salts thereof with the existing ions are used, if possible, such as NaH₂PO₄, Na₂B₄O₇, or Mg(MnO₄)₂. The conversion solution preferably contains one or more cations selected from the group consisting of K⁺, Na⁺, NH₄⁺, Ca²⁺, and Mg²⁺.

Finally, the ions MnO₄²⁻, MnO₄⁻, VO₃⁻, WO₄²⁻, MoO₄²⁻, TiO₃²⁻, ZrO₃²⁻, and NbO₄⁻, are used in the redox system as the oxidants which initiate and maintain the electrochemical procedure resulting in the formation of the conversion layer. The conversion solution preferably contains one or more anions selected from the group consisting of MnO₄²⁻, MnO₄⁻, VO₃⁻, WO₄²⁻, MoO₄²⁻, TiO₃²⁻, ZrO₃²⁻, and NbO₄⁻.

An especially preferred conversion solution contains:

(i) OH⁻;

(ii) one or more anions selected from the group consisting of PO₄³⁻, H₂PO₄⁻, HPO₄²⁻, BO₃³⁻, B₄O₇²⁻, and SiO₃²⁻ to form a cover layer;

(iii) one or more cations selected from the group consisting of K⁺, Na⁺, NH₄⁺, Ca²⁺, and Mg²⁺; and

(iv) one or more anions selected from the group consisting of MnO₄²⁻, MnO₄⁻, VO₃⁻, WO₄²⁻, MoO₄²⁻, TiO₃²⁻, ZrO₃²⁻, and NbO₄⁻ as the oxidant.

The conversion solution optionally contains buffers, in particular, alkaline buffers such as EDTA, ethylene diamine, and hexamethylene tetramine. Alkaline buffers support the formation of the barrier layer by their high content of OH— ions. Furthermore, the alkaline buffers have a favorable effect on the stability of the conversion solution.

The implant entirely or at least partially comprises the biocorrodible magnesium alloy. For purposes of the present disclosure, an alloy is a metallic structure whose main component is magnesium. For purposes of the present disclosure, the term main component is defined as the alloy component whose weight proportion of the alloy is highest. A proportion of the main component is preferably more than 50 wt. %, in particular, more than 70 wt. %.

The magnesium alloy preferably contains yttrium and further rare earth metals, because an alloy of this type is distinguished due to its physiochemical properties and high biocompatibility, in particular, also its degradation products.

A magnesium alloy of the composition rare earth metals 5.2-9.9 wt. %, thereof yttrium 3.7-5.5 wt. %, and the remainder <1 wt. % is especially preferable, magnesium making up the proportion of the alloy to 100 wt. %. This magnesium alloy has already confirmed its special suitability experimentally and in initial clinical trials, i.e., the magnesium alloy displays a high biocompatibility, favorable processing properties, good mechanical characteristics, and corrosion behavior adequate for the intended uses. For purposes of the present disclosure, the collective term “rare earth metals” is understood to include scandium (21), yttrium (39), lanthanum (57) and the 14 elements following lanthanum (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70) und lutetium (71).

The magnesium alloy is to be selected in its composition in such a way that the magnesium alloy is biocorrodible. For purposes of the present disclosure, alloys are referred to as biocorrodible when degradation occurs in a physiological environment which finally results in the entire implant or the part of the implant made of the material losing its mechanical integrity. Artificial plasma, as has been previously described according to EN ISO 10993-15:2000 for biocorrosion assays (composition NaCl 6.8 g/l, CaCl₂ 0.2 g/l, KCl 0.4 g/l, MgSO₄ 0.1 g/l, NaHCO₃ 2.2 g/l, Na₂HPO₄ 0.126 g/l, NaH₂PO₄ 0.026 g/l), is used as a testing medium for testing the corrosion behavior of an alloy being considered. For this purpose, a sample of the alloy to be assayed is stored in a closed sample container with a defined quantity of the testing medium at 37° C. At time intervals, tailored to the corrosion behavior to be expected, of a few hours up to multiple months, the sample is removed and examined for corrosion traces in a way known in the prior art. The artificial plasma according to EN ISO 10993-15:2000 corresponds to a medium similar to blood and thus represents a possibility for simulating a reproducible physioloical environment.

For purposes of the present disclosure, the term corrosion relates to the reaction of a metallic material with its environment, a measurable change the material being caused which, upon use of the material in a component, results in an impairment of the function of the component. For purposes of the present disclosure, a corrosion system comprises the corroding metallic material and a liquid corrosion medium which simulates the conditions in a physiological environment in its composition or is a physiological medium, particularly blood. On the material side, the corrosion factors influence the corrosion, such as the composition and pretreatment of the alloy, microscopic and submicroscopic inhomogeneities, boundary zone properties, temperature and mechanical tension state, and, in particular, the composition of a layer covering the surface. On the side of the medium, the corrosion process is influenced by conductivity, temperature, temperature gradients, acidity, volume-surface ratio, concentration difference, and flow velocity.

Redox reactions occur at the phase boundary between material and medium. For a protective and/or inhibiting effect, existing protective layers and/or the products of the redox reactions must implement a sufficiently dense structure, have increased thermodynamic stability in relation to the environment, and have little solubility or be insoluble in the corrosion medium. In the phase boundary, more precisely in a double layer forming the phase boundary, adsorption and desorption processes occur. The procedures in the double layer are influenced by the cathodic, anodic, and chemical partial processes occurring in the double layer. In magnesium alloys, typically a gradual alkalinization of the double layer is to be observed. Foreign material deposits, contaminants, and corrosion products influence the corrosion process. The procedures during corrosion are accordingly highly complex and may not be predicted at all or may be predicted only to a limited extent precisely in connection with a physiological corrosion medium, i.e., blood or artificial plasma, because there is no comparative data. For this reason alone, finding a corrosion-inhibiting coating, i.e., a coating which only is used for temporary reduction of the corrosion rate of a metallic material of the composition cited above in a physiological environment, is a measure outside the routine of one skilled in the art. This is particularly true for stents which are subjected to local high plastic deformations at the time of implantation. Conventional approaches using rigid corrosion-inhibiting layers are unsuitable for conditions of this type.

The procedure of corrosion may be quantified by specifying a corrosion rate. Rapid degradation is connected to a high corrosion rate and vice versa. A surface modified in the meaning of the present disclosure would result in reduction of the corrosion rate in regard to the degradation of the entire molded body. The corrosion-inhibiting coating according to the present disclosure may itself be degraded in the course of time and/or may only protect the areas of the implant covered thereby to a lesser and lesser extent. Therefore, the course of the corrosion rate is nonlinear for the entire implant. Rather, a relatively low corrosion rate results at the beginning of the occurring corrosive processes, which increases in the course of time. This behavior is understood as a temporary reduction of the corrosion rate in the meaning of the present disclosure and distinguishes the corrosion-inhibiting coating. In the case of coronary stents, the mechanical integrity of the structure is to be maintained over a period of time of three months after implantation.

The treatment in step b) is preferably performed by anodic oxidation with application of a voltage to the implant. The implant to be treated is placed in an electrically conductive liquid (electrolyte), where the implant is connected to a DC voltage source as the anode. The cathode usually comprises stainless steel, lead, aluminum or the like. Anions migrate to the implant surface in the resulting voltage field. The anions react in the voltage field with the material and a conversion layer forms. In aqueous media, hydrogen, which escapes in the form of gas, may form at the cathode. The resulting coating may also have a multilayered structure, e.g., a thin barrier layer, which is almost nonporous, extremely dense, and electrically insulating; and a much more voluminous, slightly porous cover layer, which forms by a chemical reaction of the barrier layer with the electrolyte, may be provided.

Preferably, conversion solutions which contain one or more ions selected from the group consisting of NH₄⁺, PO₄³⁻, and/or BO₃³⁻ are used as the electrolyte for the anodic oxidation with external power source.

The anodic oxidation may also be performed with an external power source under plasma discharge. The magnesium stent is electrically contacted and impinged by a high voltage of greater than 100 volts. Plasma (sparks) thus arises on the surface of the stent, by which the material surface is converted into an oxide ceramic layer.

Alternatively to anodic oxidation with an external power source, the treatment in step b) may also be performed without an external power source. The corrosion-inhibiting coating arises through redox reactions on the surface of the material. The conversion solution preferably contains one or more ions selected from the group consisting of K⁺, Na⁺, NH₄⁺, MnO₄, and VO₃⁻.

This redox reaction is reinforced by contacting the magnesium material with a noble metal in the electrolyte. A higher potential difference results due to the different electrochemical potentials of the magnesium alloy and the noble metal.

In the combination magnesium (potential −2.37 volts) with pure platinum (potential +1.60 volts), for example, the difference is 3.97 volts. This voltage is sufficient to initiate the redox reaction and produce a conversion layer in the affected electrolyte.

In an optional exemplary embodiment, in step a) of the treatment, the implant is additionally contacted with a noble metal. These noble metals preferably comprise Pt, Au, Rh, and Ru.

A second feature of the present disclosure provides an implant produced according to the method described above.

For purposes of the present disclosure, implants are devices introduced into the body via a surgical method and comprise fasteners for bones, such as screws, plates, or nails, intestinal clamps, vascular clips, prostheses in the area of the hard and soft tissue, and anchoring elements for electrodes, in particular, of pacemakers or defibrillators. The implant entirely or partially comprises the biocorrodible material. If the implant only partially comprises the biocorrodible material, this part of the implant is to be coated accordingly.

The implant is preferably a stent. Stents of typical construction have a filigree structure made of metallic struts which is first provided in a non-expanded state for introduction into the body and which is then expanded into an expanded state at the location of application. Special requirements exist for the corrosion-inhibiting layer in stents. The mechanical strain of the material during the expansion of the implant (dilation) has an influence on the course of the corrosion process, and it is to be assumed that the tension crack corrosion will be greater in the strained areas. A corrosion-inhibiting layer takes this circumstance into consideration. Furthermore, a hard corrosion-inhibiting layer may chip off during the expansion of the stent and cracking in the layer during expansion of the implant may be unavoidable. Finally, the dimensions of the filigree of metallic structure are to be noted and, if possible, only a thin, but also uniform corrosion-inhibiting layer is to be generated. It has been shown that the application of the coating according to the present disclosure entirely or at least extensively meets these requirements.

The corrosion-inhibiting coating obtainable by the treatment using the conversion solution preferably has a layer thickness in the range from 300 nm to 20 μm, in particular, in the range from 800 nm to 10 μm.

The present disclosure is described in greater detail in the following on the basis of exemplary embodiments.

### EXAMPLES

### Exemplary Embodiment 1—Anodic Oxidation Without External Power Source (Immersion Method)

Stents made of the biocorrodible magnesium alloy WE43 (93 wt. % magnesium, 4 wt. % yttrium (W), and 3 wt. % rare earth metals (E) other than yttrium) were washed using isopropanol under ultrasound and subsequently pickled for 30 seconds in 10% hydrofluoric acid. After being washed multiple times using deionized water, the stent was immersed in the wet state for 5 minutes in an aqueous conversion solution, heated to 300, of the composition 3 g/l KMnO₄ and 1 g/l NH₄VO₃. The pH value of the conversion solution was 7.5+/−0.2. After the stent was removed from the conversion solution, the implant having its brown conversion layer was washed multiple times using deionized water and then dried for 30 minutes in the circulating air dryer at 120° C.

The following experiments were performed to characterize the degradation behavior:

(i) The stents were laid at room temperature for 4 hours in artificial plasma, removed again, and judged visually in regard to the state of the degradation. (ii) The stents were stored at room temperature for 4 hours in artificial plasma. A polarization resistance was periodically detected simultaneously. (iii) The stents were stored at room temperature for 4 hours in artificial plasma. The elution rate of significant ions dissolved from the alloy was periodically ascertained from the solution. (iv) The stent was implanted in animals. A histological evaluation, μ-CT analysis, and analysis of the composition of the in vivo degraded explants followed.

### Exemplary Embodiment 2—Anodic Oxidation Without External Power Source (Immersion Method)

Stents made of the magnesium alloy WE 43 were washed using isopropanol under ultrasound and subsequently briefly wetted using demineralized water.

The wet stent was immersed in the conversion solution.

The conversion solution had the following composition:

| NaMnO₄: | 2.7 g/l |
|---|---|
| NH₄VO₃: | 1.0 g/l |
| pH: | 7.5 ± 0.2 |

Parameters for the anodic oxidation without external power source:

| | | |
|---|---|---|
| bath temperature: | 30° | C. |
| treatment time: | 10 | min |

After the stent was removed from the conversion solution, it was washed multiple times and subsequently dried for 30 minutes at 120° C.

Exemplary Embodiment 3—Anodic Oxidation with External Power Source

Stents made of the biocorrodible magnesium alloy WE 43 were washed using isopropanol under ultrasound and subsequently briefly wetted using demineralized water.

The wet stent was connected as the anode and introduced into a conversion electrolyte.

The electrolyte had the following composition:

| NaMnO₄: | 2.7 g/l |
|---|---|
| NH₄VO₃: | 1.0 g/l |
| pH: | 7.5 ± 0.2 |

The parameters of the anodic oxidation with external power source were:

| | | |
|---|---|---|
| voltage: | 5 | V |
| bath temperature: | 30° | C. |
| treatment time: | 5 | min |
| current: | 20 | mA |

After the anodization, the stent was washed well in demineralized water and dried for 30 minutes at 120° C.

The conversion layer obtained was 2 to 3 μm thick.

Exemplary Embodiment 4—Anodic Oxidation in Contact with Noble Metals

Stents made of the biocorrodible magnesium alloy WE 43 were pretreated as in exemplary embodiment 3; the conversion electrolyte had the same composition as in exemplary embodiment 3.

The stent was contacted fixed on a circuit board and immersed in the conversion solution.

Parameters for the anodic oxidation in contact with noble metals:

| | | |
|---|---|---|
| bath temperature: | 30° | C. |
| treatment time: | 10 | min |

The post-treatment of the coated stent was performed in the same way as in exemplary embodiment 3.

The conversion layer obtained was approximately 2 μm thick.

Exemplary Embodiment 5—Anodic Oxidation with External Power Source Under Plasma Discharge in the Electrolyte

Stents made of the biocorrodible magnesium alloy WE 43 were pretreated as in exemplary embodiment 3.

The wet stent was connected as the anode and introduced into an aqueous conversion electrolyte of the following composition:
0.52 mol/l PO₄³⁻
0.57 mol/l BO₃³⁻
0.40 mol/l NH₄⁺
2.5 mol/l hexamethylene tetramine

The electrolyte had a pH value of 7.2.

The parameters of the anodic oxidation under plasma discharge were:

| | | |
|---|---|---|
| current density: | 1-1.4 | A/dm² |
| bath temperature: | 37-40° | C. |
| voltage: | limited to 340 | V |

After 5 minutes exposure time, the layer on the stent had a thickness of approximately 5 ∥m.

The stent was washed using demineralized water and dried.

All patents, patent applications and publications referred to herein are incorporated by reference in their entirety.

What is claimed is:

1. A method for producing a corrosion-inhibiting coating on an implant having a surface and made of a biocorrodible magnesium alloy, the method comprising:
a) treating the implant surface using an aqueous or alcoholic conversion solution comprising one or more ions selected from the group consisting of K⁺, Na⁺, NH₄⁺, Ca²⁺, Mg²⁺, Zn²⁺, Ti⁴⁺, Zr⁴⁺, Ce^{3 +}, Ce⁴⁺, PO₄³⁻, HPO₄²⁻, H₂PO₄⁻, OH⁻, BO₃³⁻, B₄O₇²⁻, SiO₃²⁻, MnO₄^{2-, MnO}₄⁻, VO₃⁻, WO₄²⁻, MoO₄²⁻, TiO₃²⁻, Se²⁻, ZrO₃²⁻, and NbO₄⁻,
wherein the concentration of the ion or the ions is in the range of from 0.01 mol/l to 2 mol/l, respectively.

2. The method of claim 1, wherein the conversion solution contains OH— ions and one or more ions selected from the group consisting of K⁺, Na⁺, NH₄⁺, Ca²⁺, Mg²⁺, Zn²⁺, Ti⁴⁺, Zr⁴⁺, Ce³⁺, Ce⁴⁺, PO₄³⁻, HPO₄²⁻, H₂PO₄⁻, OH⁻, BO₃³⁻, B₄O₇²⁻, SiO₃²⁻, MnO₄²⁻, MnO₄⁻, VO₃⁻, WO₄²⁻, MoO₄²⁻, TiO₃²⁻, Se²⁻, ZrO₃²⁻, and NbO₄⁻.

3. The method of claim 1, wherein the conversion solution contains one or more cations selected from the group consisting of K⁺, Na⁺, NH₄⁺, Ca²⁺, and Mg²⁺.

4. The method of claim 1, wherein the conversion solution contains one or more anions selected from the group consisting of MnO₄²⁻, MnO₄⁻, VO₃⁻, WO₄²⁻, MoO₄²⁻, TiO₃²⁻, ZrO₃²⁻, and NbO₄⁻.

5. The method of claim 1, wherein the conversion solution comprises:
(i) OH⁻;
(ii) one or more anions selected from the group consisting of PO₄³⁻, H₂PO₄⁻, HPO₄²⁻, BO₃³⁻, B₄O₇²⁻, and SiO₃²⁻ to form a cover layer;
(iii) one or more cations selected from the group consisting of K⁺, Na⁺, NH₄⁺, Ca²⁺, and Mg²⁺; and
(iv) one or more anions selected from the group consisting of MnO₄²⁻, MnO₄⁻, VO₃⁻, WO₄²⁻, MoO₄²⁻, TiO₃²⁻, ZrO₃²⁻, and NbO₄⁻ as oxidant.

6. The method of claim 1, wherein the treatment in step a) is performed by anodic oxidation with application of a voltage to the implant.

7. The method of claim 6, wherein the conversion solution used for anodic oxidation contains one or more ions selected from the group consisting of NH₄⁺, PO₄³⁻, and BO₃³⁻ and wherein the anodic oxidation is performed with external power source under plasma discharge.

8. The method of claim 7, wherein the conversion solution contains one or more ions selected from the group consisting of K⁺, Na⁺, NH₄⁺, MnO₄³⁻, and VO₃⁻.

9. The method of claim 1, wherein the treatment in step a) comprises contacting the implant with a noble metal selected from the group consisting of Pt, Au, Rh, and Ru.

10. An implant having a corrosion-inhibiting coating provided by a method, comprising:
a) treating the implant surface using an aqueous or alcoholic conversion solution containing one or more ions selected from the group consisting of K⁺, Na⁺, NH₄⁺, Ca²⁺, Mg²⁺, Zn²⁺, Ti⁴⁺, Zr⁴⁺, Ce³⁺, Ce⁴⁺, PO₄³⁻, HPO₄^{2 -}, H₂PO₄⁻, OH⁻, BO₃³⁻, B₄O₇²⁻, SiO₃²⁻, MnO₄²⁻, MnO₄⁻, VO₃⁻, WO₄²⁻, MoO₄²⁻, TiO₃²⁻, Se²⁻, ZrO₃²⁻, and NbO₄⁻,
wherein the concentration of the ion or the ions is in the range of from 0.01 mol/l to 2 mol/l, respectively.

11. The implant of claim 10, wherein the corrosion-inhibiting coating has a layer thickness in the range of from 300 nm to 20 μm.

12. The method of claim 2, wherein the conversion solution contains one or more cations selected from the group consisting of K⁺, Na⁺, NH₄⁺, Ca²⁺, and Mg²⁺.

13. The method of claim 1, wherein the treatment in step a) is performed by immersing the implant in the conversion solution.

14. The implant of claim 10, wherein the implant is a stent.
